(19) United States
(12) Patent Application Publication
(10) Pub. No.: US 2023/0263431 A1
(43) Pub. Date: Aug. 24, 2023

(54) METHODS, SYSTEMS, AND COMPUTER READABLE MEDIA FOR ANALYZING RESPIRATORY KINEMATICS

(71) Applicant: University of Virginia Patent Foundation, Charlottesville, VA (US)

(72) Inventors: Shrirang M. Gadrey, Crozet, VA (US); Ronald Dean Williams, Charlottesville, VA (US); Sarah J. Ratcliffe, Charlottesville, VA (US); J. Randall Moorman, Keswick, VA (US); William B. Ashe, Charlottesville, VA (US)

(21) Appl. No.: 18/018,469

(22) PCT Filed: Jul. 30, 2021
(86) PCT No.: PCT/US2021/043941

§ 371 Date: Jan. 27, 2023

Related U.S. Application Data
(60) Provisional application No. 63/058,871, filed on Jul. 30, 2020.

(51) Int. Cl. A61B 5/113; A61B 5/00

(52) U.S. Cl. CPC A61B 5/113; A61B 5/0002; A61B 5/6823; A61B 5/7275; A61B 2562/0219

(57) ABSTRACT

A method for analyzing respiratory kinematics includes collecting a plurality of kinematic signal data streams from each of a respective plurality of inertial sensor devices applied to a subject, wherein the kinematic signal data streams are synchronized with each other, transforming the plurality of kinematic signal data streams into a respective plurality of analytic signals, determining landmark points associated with each of the plurality of analytic signals to identify individual breathing intervals associated with each of the plurality of inertial sensor devices, and analyzing two or more of the individual breathing intervals to establish a magnitude-synchronicity relationship that is utilized to determine a probability of a presence of a respiratory condition existing in the subject.

### CROSS REFERENCE TO RELATED APPLICATIONS

This application claims benefit of U.S. Provisional Application Serial No. 63/058,871 filed on Jul. 30, 2020, the disclosure of which is incorporated herein by reference in its entirety.

### TECHNICAL FIELD

The presently disclosed subject matter relates to systems and techniques for measuring breathing patterns. The presently disclosed subject matter further relates to methods of remote monitoring and measuring of breathing motion patterns via the use of unobtrusive wearable sensors.

### BACKGROUND

Features of breathing motion (respiratory kinematics) like breath intervals, overall depth of breathing and the magnitude-synchrony relationships between key anatomical locations contain useful information about the subject’s health. In many clinical scenarios, breath-to-breath variability of motion patterns is also an important feature. Labored breathing patterns like abdominal paradox (inward abdominal motion, asynchronous with rib cage expansion), for example, suggests inspiratory muscle overload and is a risk marker for imminent acute respiratory failure. Ataxic breathing patterns (increased variability in interval and depth of breaths) can provide early warnings of an opiate overdose. Presently, respiratory rate is the only breathing motion pattern that can be monitored remotely. Other physical examination signs of abnormal breathing can only be visualized at the patient’s bedside. This is problematic for several reasons. First, individuals can develop severely deranged breathing patterns with minimal abnormality in respiratory rate. Second, physical examination is reported subjectively and with poor inter-rater reliability. Notably, an inexperienced caregiver may miss red-flag signs or fail to recognize the risk they portend. Moreover, timely detection relies on frequent bedside assessments by experienced clinicians, which is often not possible due to limited resources and/or staffing.

Accordingly, there is an ongoing need for an improved method and system for analyzing respiratory kinematics.

### SUMMARY

A method for analyzing respiratory kinematics includes collecting a plurality of kinematic signal data streams from each of a respective plurality of inertial sensor devices applied to a subject, wherein the kinematic signal data streams are synchronized with each other, transforming the plurality of kinematic signal data streams into a respective plurality of analytic signals, determining landmark points associated with each of the plurality of analytic signals to identify individual breathing intervals associated with each of the plurality of inertial sensor devices, and analyzing two or more of the individual breathing intervals associated with at least two of the inertial sensor devices to establish a magnitude-synchronicity relationship that is utilized to characterize breathing motion patterns exhibited in the subject.

According to another aspect of the method described herein, signal noise is removed from the kinematic signal data streams via one or more filters prior to transforming into the analytic signals.

According to another aspect of the method described herein, each of the plurality of inertial sensor devices is positioned in a unique and separate location on the torso of the subject.

According to another aspect of the method described herein, an instantaneous phase angle from each of the analytic signals is used to determine the landmark points.

According to another aspect of the method described herein, the magnitude-synchronicity relationship is provided as input to a statistical model that is configured to generate the probability.

According to another aspect of the method described herein, each of the plurality of kinematic signal data streams is collected via wireless communications.

According to another aspect of the method described herein, the magnitude-synchronicity relationship is defined by at least a comparison of magnitudes of motion exhibited by the analytic signals associated with two or more inertial sensor devices.

According to another aspect of the method described herein, the breathing motion patterns are utilized to determine a probability of a presence of a respiratory condition existing in the subject.

According to another aspect of the method described herein, the landmark points are utilized to derive a respiratory rate time series.

According to another aspect of the method described herein, identifying individual breathing intervals includes identifying breathing intervals on a breath by breath basis or on a continuous signal strip basis.

According to another aspect of the method described herein, the magnitude-synchronicity relationship is defined by a quantification of the degree of synchronicity and phase relationships exhibited by the analytic signals associated with two or more inertial sensor devices.

According to another aspect of the subject matter described herein, a system for analyzing respiratory kinematics includes a plurality of inertial sensor devices applied to a subject and a monitoring platform device including at least one processor and a memory. The system further includes an ARK engine stored in the memory and implemented by the at least one processor that is configured for collecting a plurality of kinematic signal data streams from each of the plurality of inertial sensor devices, wherein the kinematic signal data streams are synchronized with each other, transforming the plurality of kinematic signal data streams into a respective plurality of analytic signals, determining landmark points associated with each of the plurality of analytic signals to identify individual breathing intervals associated with each of the plurality of inertial sensor devices, and analyzing two or more of the individual breathing intervals associated with at least two of the inertial sensor devices to establish a magnitude-synchronicity relationship that is utilized to characterize breathing motion patterns exhibited in the subject.

According to another aspect of the system described herein, each of the plurality of inertial sensor devices is positioned in a unique and separate location on the torso of the subject.

According to another aspect of the system described herein, an instantaneous phase angle from each of the analytic signals is used to determine the landmark points.

According to another aspect of the system described herein, the magnitude-synchronicity relationship is provided as input to a statistical model that is configured to generate the probability.

According to another aspect of the system described herein, each of the plurality of kinematic signal data streams is collected via wireless communications.

According to another aspect of the system described herein, the magnitude-synchronicity relationship is defined by at least a comparison of magnitudes of motion exhibited by the analytic signals associated with two or more inertial sensor devices

According to another aspect of the system described herein, the breathing motion patterns are utilized to determine a probability of a presence of a respiratory condition existing in the subject.

According to another aspect of the system described herein, the landmark points are utilized to derive a respiratory rate time series.

According to another aspect of the system described herein, identifying individual breathing intervals includes identifying breathing intervals on a breath by breath basis or on a continuous signal strip basis.

According to another aspect of the method described herein, the magnitude-synchronicity relationship is defined by a quantification of the degree of synchronicity and phase relationships exhibited by the analytic signals associated with two or more inertial sensor devices.

The subject matter described herein may be implemented in hardware, software, firmware, or any combination thereof. As such, the terms “function” “engine” or “module” as used herein refer to hardware, which may also include software and/or firmware components, for implementing the feature being described. In one exemplary implementation, the subject matter described herein may be implemented using a computer readable medium having stored thereon computer executable instructions that when executed by the processor of a computer control the computer to perform steps. Exemplary computer readable media suitable for implementing the subject matter described herein include non-transitory computer-readable media, such as disk memory devices, chip memory devices, programmable logic devices, and application specific integrated circuits. In addition, a computer readable medium that implements the subject matter described herein may be located on a single device or computing platform or may be distributed across multiple devices or computing platforms.

### BRIEF DESCRIPTION OF THE DRAWINGS

The subject matter described herein will now be explained with reference to the accompanying drawings of which:

FIG. 1 is an exemplary system architecture for analyzing respiratory kinematics according to an embodiment of the subject matter described herein;

FIG. 2 is a diagram depicting an exemplary sensor device placement on a subject torso according to an embodiment of the subject matter described herein;

FIG. 3 depicts a plurality of graphs illustrating the selection of landmark points during a resting stage according to an embodiment of the subject matter described herein;

FIG. 4 depicts a plurality of graphs illustrating the selection of landmark points during an exhaustion stage according to an embodiment of the subject matter described herein;

FIG. 5 depicts a plurality of accelerometer signals obtained from a plurality of sensor devices at different respiratory stages according to an embodiment of the subject matter described herein;

FIG. 6 depicts a plurality of plots illustrating respiratory rate sampling according to an embodiment of the subject matter described herein;

FIG. 7 depicts a plurality of graphs illustrating kinematics-flow coupling measurements according to an embodiment of the subject matter described herein;

FIG. 8 depicts a plurality of graphs illustrating the diversity of observed respiratory rate signals according to an embodiment of the subject matter described herein;

FIG. 9 depicts a plurality of plots illustrating kinematics-flow coupling measurement comparisons of two individuals according to an embodiment of the subject matter described herein;

FIG. 10 depicts a bar chart illustrating entropy across various individuals and stages according to an embodiment of the subject matter described herein; and

FIG. 11 is a flow chart illustrating an exemplary process for analyzing respiratory kinematics according to an embodiment of the subject matter described herein.

### DETAILED DESCRIPTION

Systems and methods for analyzing respiratory kinematics are described herein. In particular, the disclosed subject matter pertains to an Analysis of Respiratory Kinematics (ARK) system that employs unobtrusive wearable sensor devices to reliably detect and quantify breathing motion patterns exhibited in a patient subject. Notably, tests conducted among healthy volunteer subjects have demonstrated that ARK metrics were strongly associated with exercise-induced respiratory muscle overload. Further, this technology has also been validated among emergency room (ER) patients with an acute respiratory illness consistent with COVID-19 and other breathing-related disorders.

Labored breathing is an example of abnormal breathing motion pattern which signifies respiratory muscle overload and it is a strong, early predictor of respiratory failure. Another example of a clinically significant breathing motion pattern is the typical ataxic pattern that is a risk marker for opiate overdoses. Such breathing motion patterns can be recognized by visualizing certain aberrations in the breath related motion of the ribs and abdomen (i.e., respiratory kinematics). Features of interest include breath intervals, overall depth of breathing and the magnitude-synchrony relationships between key anatomical locations. These features contain information about the subject’s health which can be useful in wide range of respiratory and non-respiratory illnesses. Common respiratory kinematic signatures of labored breathing include an increase in contraction of neck muscles to augment upper rib-cage expansion, respiratory alternans (i.e., rib-predominant breaths alternating with abdomen-predominant breaths), and abdominal paradox (i.e., inward abdominal motion exhibited during rib expansion). In opiate induced ataxic breathing, short breaths alternate with long ones or with apnea (breath interval variability), shallow breaths alternate with deeper ones (tidal volume variability) and thorax-predominant breaths alternate with abdomen-predominant or mixed ones (respiratory alternans). Tachypnea (short breath interval) and hyperventilation (increased depth of breaths) in the setting of infection can herald the onset of sepsis.

Moreover, deranged breathing motion patterns (e.g. labored breathing in a COVID-19 patient or ataxic breathing in a patient receiving opiate pain medicines) indicate a high risk of rapid clinical deterioration and should always prompt immediate medical attention. Specifically, deranged breathing motion patterns can only be visualized at the patient’s bedside and there is presently no way to remotely monitor for it. Of all the remotely monitored parameters, only respiratory rates (RR) can be reasonably considered to be an indirect marker for abnormal breathing. However, this parameter and the manner of monitoring the same is far from reliable. As an example, frail and vulnerable patients (e.g., nursing home residents) can develop severely labored breathing without tachypnea (high RR). Similarly, patients can develop severely ataxic breathing from opiates without bradypnea (low RR). Studies have demonstrated that only a small proportion of the variance in severity of breathing motion derangement is explained by routinely monitored metrics. These problems are only exacerbated by the unique barriers existing in bedside assessments pertaining to COVID-19 patients (e.g., health system overload, risk of staff exposures, personal protective equipment shortages, etc.). Without frequent bedside assessments, deranged breathing motion may go undetected in COVID-19 or other patients. As such, the resulting delays in treatment may prove fatal for many vulnerable patients. Accordingly, it is clear that better methods for remote respiratory monitoring are urgently needed.

As indicated above, the disclosed subject matter pertains to an ARK system that yields new methods for measuring and monitoring breathing motion derangements. The ARK system may be configured to record respiratory motion signals using very small but powerful motion sensor devices (e.g., microelectromechanical inertial measurement units (MEMS-IMUs) placed on key anatomical positions of the chest and abdomen. Notably, key features of these signals are measured to create a collection of novel vital signs (“ARK metrics”) that represent breathing motion patterns. Based on some studies, the collection and analysis of remotely monitored ARK metrics could ensure early detection of deranged breathing and save lives by bringing immediate attention to deteriorating patients.

In many instances, conventional monitoring systems only allow for RR monitoring. Some devices offer both RR and tidal volume (VT) monitoring using impedance (e.g., Respiratory Motion Inc.), acoustic (e.g., RTM Vital Signs) signals, or inductance plethysmography. However, no existing system is able to monitor deranged breathing motion patterns. This is an immense clinical advantage since the breathing motion pattern is often a better predictor of respiratory instability and collapse than using RR and/or VT. Other methods to improve detection of deranged breathing motion have included standardized mnemonics or ordinal rating scales. While such methods may improve the uniformity of reporting, they remain reliant on frequent bedside assessments. Further, the sole objective metric utilized by the aforementioned conventional monitoring systems is the respiratory rate. All other signs of imminent ventilatory failure (i.e., hyper-carbic respiratory failure), like “working hard to breathe” or “using accessory muscles”, are subjectively judged by visual inspection of the patient’s respiratory motion.

In contrast, the disclosed ARK system is the only approach that simultaneously affords (a) detection of breathing motion pattern in addition to RR and VT, (b) fully quantitative reporting compatible with advanced predictive modelling, and (c) remote objective monitoring to eliminate reliance on bedside assessments.

In particular, the ARK system can be configured to detect deranged breathing patterns (e.g., like accessory respiratory muscle use or rib-abdomen asynchrony) and report these patterns as quantitative vital signs that can be used in predictive modelling of imminent respiratory collapse.

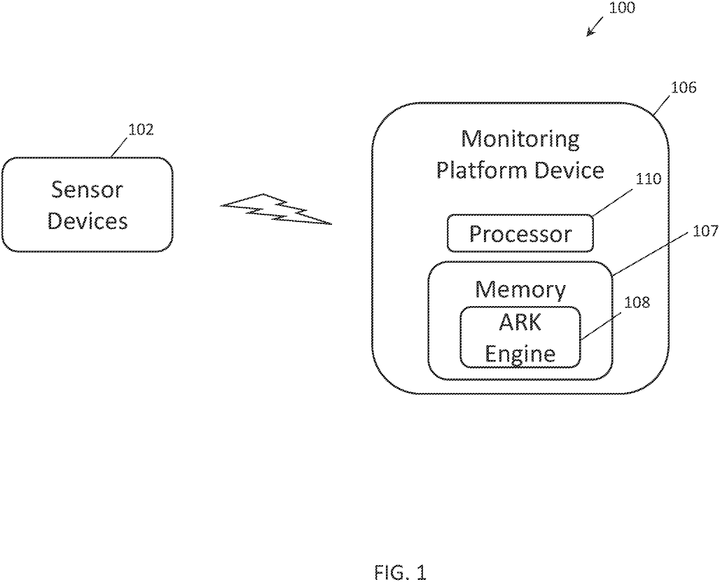

FIG. 1 illustrates an exemplary ARK system configured to analyze respiratory kinematics to detect deranged breathing motion patterns. As shown in FIG. 1, system 100 includes a plurality of inertial sensor devices 102 and a monitoring platform device 106. Notably, monitoring platform device 106 includes and supports an ARK engine 108, which may comprise a software algorithm that is stored in memory 107 and executed by one or more hardware processing units 110 of monitoring platform device 106. In some embodiments, monitoring platform device 106 may comprise any computing device, such as a personal computer, a laptop computer, a smartphone device, a tablet device, and the like. In particular, monitoring platform device 106 is configured to receive signaling data that is captured by inertial sensor devices 102. Notably, the signaling data can be communicated by inertial sensor devices 102 to monitoring platform device 106 via either wireless communications or a wired communications means. In some embodiments, inertial sensor devices 102 can include battery powered devices that are equipped with radio frequency (RF) circuitry components. Similarly, monitoring platform device 106 may include a similar chipset component that is capable of conducting wireless communications with sensor devices 102 (e.g., WiFi, Bluetooth, or any other suitable wireless standard).

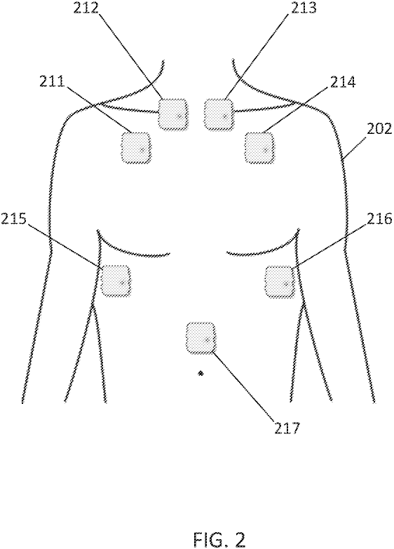

Each of the inertial sensor devices 102 can also include a plurality of sensor elements, such as, accelerometers, gyroscopes (e.g., angular velocity in XYZ coordinate space), magnetometers, and the like. While inertial sensor devices 102 can be configured to process a plurality of different data streams signals without departing from the disclosed subject matter, the following disclosure describes the measurement of a linear acceleration signals made by each of sensor devices 102. Further, sensor devices 102 can also include adhesive tape that can be used to attach to a subject’s chest and abdomen. For example, inertial sensor devices 102 may be placed on and temporarily adhered to a patient subject in a manner as depicted in FIG. 2. In some embodiments, each sensor device 102 may have its own processor that can collect and store some amount of data in local memory.

FIG. 2 depicts an exemplary placement configuration of sensor devices on a patient torso. For example, FIG. 2 illustrates the position and placement of a plurality of sensor devices 211-217 on the torso of a patient subject 202. Although FIG. 2 depicts the approximate position of seven (7) sensor devices, any manner of placement and/or any number of sensor devices can be utilized without departing from the scope of disclosed subject matter. As shown in FIG. 2, the topmost sensor devices (e.g., 212-213) may be placed at the insertion of sternocleidomastoid, near the sternoclavicular joint of subject 202. The second rib sensors 211 and 214 (i.e., sensor devices placed on the second rib) can be placed in the mid-clavicular line and the eighth rib sensors (215-216) (i.e., sensor devices placed on the eighth rib) may be placed in the anterior axillary line. Further, an abdominal sensor device 217 can be placed in the midline abdomen, at that spot above the umbilicus where respiratory motion is most prominently visible to the technician. In some embodiments, a sensor device can also be placed at the base of the neck in the posterior midline (not shown) to capture non-respiratory motions of the torso. In some embodiments (e.g., in a clinical setting), resting data may be obtained in 2-minute recordings from of all sensor locations with patient subjects placed in a supine position, e.g., with 30 degree head elevation.

In order to properly configure the disclosed ARK system to objectively measure respiratory kinematics, each of the plurality of inertial sensor devices requires synchronization. For example, each of the plurality of inertial sensors shown in FIG. 2 can be connected via universal serial bus (USB) to a host application (e.g., ARK engine 108) running on a computer device, such as monitoring platform device 106. Data collection on the sensors may be stagger-started over eight consecutive ticks of a common 100 Hertz (Hz) timer on the monitoring platform device 106. Once started, each sensor device can record signals at a sampling frequency of 100 Hz based on a local timer on that same sensor device. Data can be realigned to a common time frame using the initial host clock start time (e.g., supported by platform device 106 and/or ARK engine 108) and the stagger order of the sensor devices. Due to minor inconsistencies between local timers on the sensor devices, the data can be interpolated to identical sampling times across all of the sensor devices. For interpolation, data may be up-sampled to a common ten (10) microsecond clock by duplicating samples and filtering, then decimated to fall on hundredth-second boundaries.

In some embodiments, the synchronization of the inertial sensor devices can be conducted via wired connections (e.g., with each other and/or the monitoring platform device). Notably, a wired connection and associated hardware may produce consistent transmission delays, with some zero-mean, additive random noise. One exemplary synchronization process may include calculating an average transmission delay associated with One Way Time of Flight processing. Initial preparation includes connecting one or more sensor devices to master device. The synchronization process further includes executing a loop, whereby a master processor sends a “timing” command to a sensor device in order to simultaneously start a timer. The sensor device may start the timer upon receipt, processes commands, and prepares “timing response” message. In some embodiments, the sensor device is configured to wait until the transmission medium is ready, then stops the timer. Notably, the timer result now contains the time used in processing (i.e., the amount of time not in flight). The sensor device immediately puts the processing time into a message that is sent back to the master device. Upon receipt of the message, the master device stops the timer and subtracts the processing time in order to determine the total Time of Flight value. Dividing by two produces an estimate of the transmission delay. Repeating and averaging to find a consistent estimate of transmission delay is subsequently conducted. At this stage of the process, the ARK engine repeats the above steps for the remaining devices.

In particular, the ARK engine estimates the sensor device time’s periodic frame of the master device’s recurring timer. The next step involves the master device having a recurring (Tₛₐₘₚ, i.e., 10 millisecond period) timer to determine an ideal data-collection frame. Moreover, a sensor device has a much faster recurring timer to count ticks of time (T_{Dclock}, i.e., 10 microsecond period). The goal for synchronization is that out of every

$$\frac{{T}_{s a m p}}{{T}_{D c l o c k}}$$

ticks (i.e. 1000 ticks) on the sensor device, the single recurring frame closest to master’s timer tick is found. Further, the master device sends a transmission delay to the sensor device.

A loop is then constructed when the master device sends a “run” message. For example, the sensor device records local time of receipt in clock ticks, subtracts transmission delay to find estimate of send time, applies a modulus function to find the periodic estimate of send time, and applies an iterative circular averaging method to find running periodic average. This process is repeated by the ARK engine until there are enough measurements such that maximum single change is below an acceptable level (e.g., 256 times). The result is a device-specific alignment of a sensor device timer frame to a master sensor device timer frame. The process is subsequently repeated for the remaining sensor devices.

In some embodiments, the ARK engine initiates the data collection stage by assigning indices for each sensor device. For example, the ARK engine starts with a first sensor device (i.e., sensor device “1”) and executes the following function on a loop. On the next master device timer tick, the ARK engine sends a “Run message” to the first sensor device. Notably, the first sensor device will receive the message after the sending tick but before the next tick, with sufficient time to arm its data acquisition functionality for collection starting on the next tick. The ARK engine repeats this process by looping through all of the indexed sensor devices in order, starting n sensors in n consecutive ticks. The ARK engine will also record a real-clock master time of the start of the first sensor device and the start order of the sensor devices for future realignment once all data has been recorded to local memory.

In some embodiments, the synchronization of the inertial sensor devices can be conducted via wireless communications (e.g., with each other and/or the monitoring platform device). Utilizing this mode of synchronization further assumes that the wireless medium and radios produce consistent transmission delays with some zero-mean, additive random noise. It is also assumed that the average difference between response times of different sensor devices is minimal. In some embodiments, the ARK engine is configured (after a wireless connection is established) to send a start data command to each of the sensor devices, which in turn triggers each of the sensor devices to start collecting data. The data collection process can be repeated and/or restarted at the end of the data collection period. Notably, the ARK engine is configured to adjust the start times to be the same by adding offsets to the entire time vector of each sensor device. Further, the ARK engine may average the end times of the device data collection periods to find a common end time. Afterwards, the ARK engine can be configured to apply a linear transformation to adjust (e.g., stretch or shrink) the time vector to simultaneously set a start time and end time as well as to adjust other times accordingly. The ARK engine may subsequently apply a resampling process to re-align the collected data. In addition, the ARK sensor is further configured, via a loop for all of the sensor devices, to i) find a closest rational interpolation factor that would adjust the measured data rate to a desired data rate, which is bounded by some computational maximum (e.g., maximum up-sample factor of 2000), ii) up-sample by numerator of rational, iii) select a new starting index to align the data frames, and iv) decimate by denominator of rational.

After the synchronization process is completed, monitoring platform device 106 and/or ARK engine 108 can be configured to conduct a signaling filtering process on data signals received from the plurality of inertial sensor devices. For example, monitoring platform device 106 can be configured to filter the accelerometer signals (e.g., kinematic signaling data and/or data streams) to preserve the respiratory content and to reduce the amplitude of the non-respiratory components. In some embodiments, monitoring platform device 106 and/or ARK engine 108 may use a Butterworth bandpass filter (e.g., a fourth order low pass and sixth order high pass; zero-phase non-causal filter) with corner frequencies of 0.05 Hz and 1 Hz. Notably, this example design choice is based on the fact that the frequency of human respirations can reasonably be expected to range between the corresponding rates of 3 and 60 breaths per minute in most circumstances, including after maximal exercise.

After filtering the accelerometer signal data, monitoring platform device 106 and/or ARK engine 108 may be further configured to process the filtered data in order identify and designate individual breath intervals and/or landmark points existing on the accelerometer signals. For example, using the filtered signals (e.g., acceleration signal data), ARK engine 108 can generate the corresponding analytic representations (e.g., analytic signals). For any real valued signal u(t), its analytic representation uₐ(t) is defined as:

$${u}_{a} \left(t\right) ≜ u \left(t\right) + i ⋅ H \left(u\right) \left(t\right)$$

Here, H(u)(t) is the Hilbert transform of u(t), which shifts the phase of its components by π/2 radians for negative frequencies and by -π/2 radians for positive frequencies. Notably, the phase angles from the analytic representation can be used by ARK engine 108 to identify the landmark points on the accelerometer signals. In the analytic representation, the Hilbert transform can be plotted on the imaginary y-axis [i·H(u)(t)] as a function of the untransformed signal in the real x-axis of a complex plane. Multiplying by i shifts all phases by an additional π/2 radians, thereby restoring the phase of positive frequency components while negating the negative frequency components when added to the original, real signal.

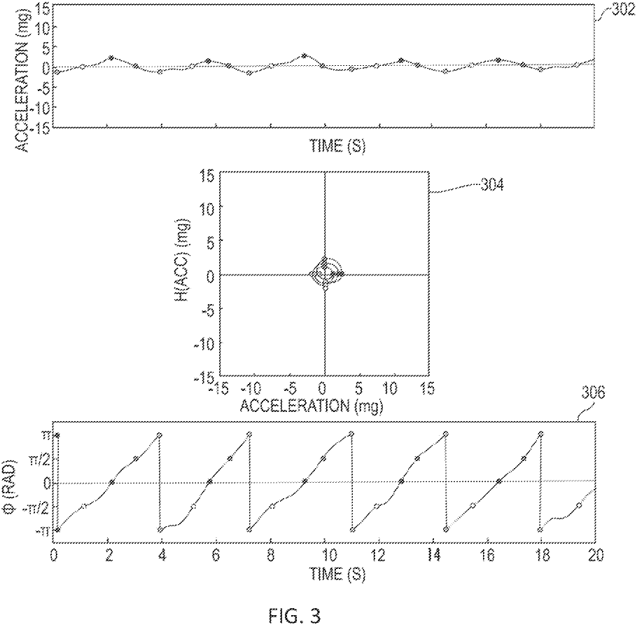

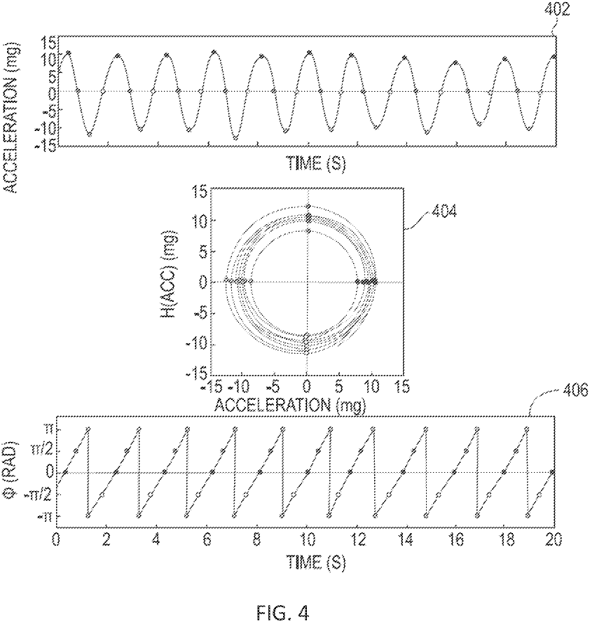

The angle of the analytic signal with the positive real axis in the complex plane may represent the time-varying phase of the signal wave. Since the analytic representation contains only positive frequencies, the resulting instantaneous phase angle (φ) of the wave monotonically increases to match the progression of the complex analytic signal around the origin. For quasi-cyclic processes with repetitive but not strictly periodic behavior, this instantaneous phase angle can be used to reliably detect consistent landmarks, where a given phase closely tracks the same point on the original signal (e.g., a specific peak or zero-crossing) across different cycles). In some embodiments, ARK engine 108 uses this property to identify breath intervals on accelerometer signals as the intervals between successive occurrences of a particular phase angle. For example, FIGS. 3 and 4 illustrate a plurality of graphs used to select landmark points using the phase angle from the analytic representation of the signal. Notably, graphs 302-306 in FIG. 3 depict signals collected while a subject is rested and graphs 402-406 show signals collected at exhaustion from the same subject. In particular, graphs 302 and 402 show the acceleration signal (i.e., kinematic signal data stream) in time domain. Similarly, graphs 304 and 404 show the corresponding analytic representations of the aforementioned acceleration signals. In an analytic representation of a signal (e.g., analytic signal), the Hilbert transform of the signal is plotted, on an imaginary y-axis of a complex plane, as a function of the untransformed signal. In this complex plane, the phase angle for any number is defined as angle between the positive real axis and the line joining the origin and that number. Graph 306 in FIG. 3 and graph 406 in FIG. 4 each shows a time domain plot of the instantaneous phase angle. Across all panels, the points with phase angles of 0, 0.5π, π and -0.5π radians are indicated accordingly. More specifically, the different dots demonstrate the property that each occurrence of a particular phase angle is separated from the last by one respiratory cycle. It is also noted that points of maxima and minima in the untransformed signal correspond with the phase angles of 0 and π on the analytic representation. Similarly, zero crossings on the untransformed signal correspond with phase angles

$$\text{of} \frac{π}{2} \text{and} - \frac{π}{2}$$

on the analytic representation. For flow signals (e.g., in a laboratory setting), these points are clinically significant and represent onset of inhalation

$$\left(\frac{π}{2}\right) ,$$

onset of exhalation

$$\left(- \frac{π}{2}\right) ,$$

peak inspiratory flow rate (0), and peak expiratory flow rate (π) respectively.

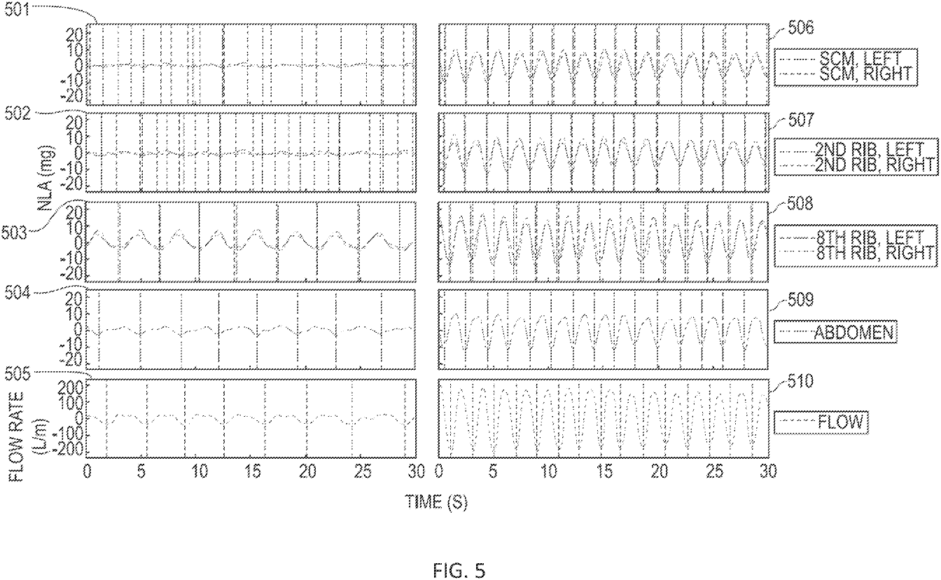

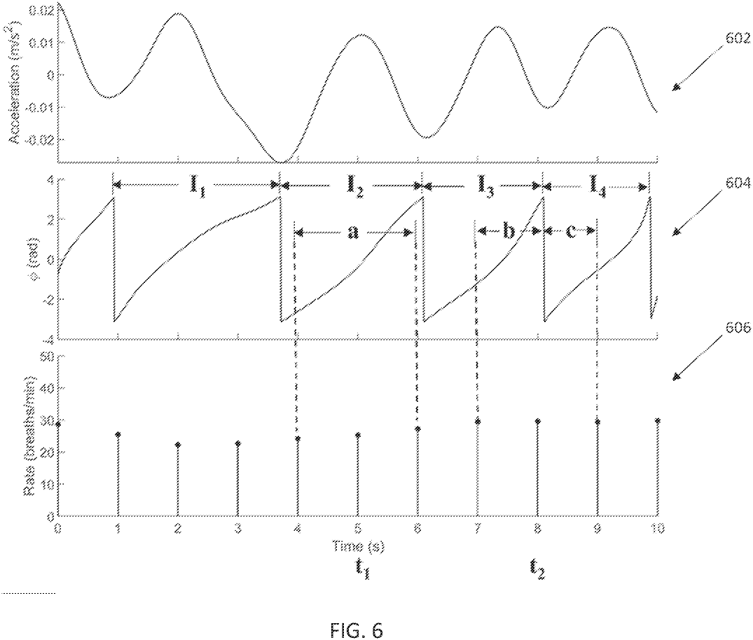

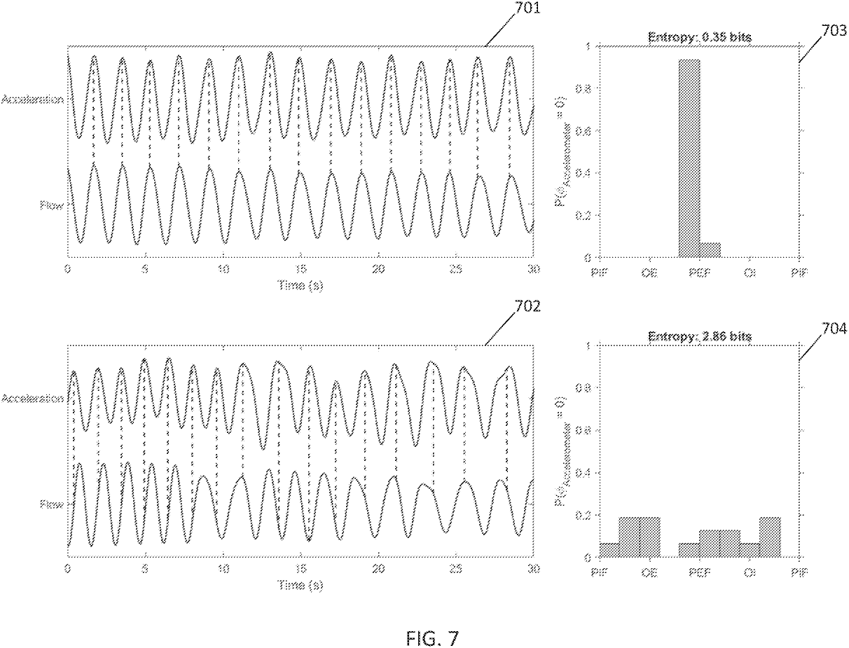

The aforementioned synchronized kinematic signal stream data and determined landmarks can be visualized in FIG. 5. For example, FIG. 5 shows 30 second strips of accelerometer signals obtained at rest (e.g., graphs 501-504) and after maximal exercise (e.g., graphs 506-509) by the same individual. The top 8 graphs are organized by kinematic sensor device location: sternocleidomastoid insertion (SCM) (graphs 501 and 506), 2nd rib (graphs 502 and 507), 8th ribs (graphs 503 and 508), and midline abdomen (graphs 504 and 509), respectively. Graphs 505 and 510 show air flow signals recorded using exercise laboratory equipment. Notably, the vertical lines mark the location of landmark points selected using a phase angle of π radians. The intervals between these landmarks decrease sharply, reflecting tachypnea after exertion. Additionally, significant changes can be noted in magnitude and synchrony of motion at various sensor locations. Most prominently, thorax predominant breathing at rest changes to a mixed thoraco-abdominal breathing at exhaustion (comparing signals at 8th rib and abdomen). Upper thoracic motion (SCM and 2nd rib), which likely reflects accessory respiratory muscle recruitment, rises from being negligible to being comparable to lower thoracic signals at exhaustion. In all the graphs that capture a meaningful degree of respiratory motion, the interval between these landmarks was defined as the accelerometer-derived breath interval. These intervals were used to derive a respiratory rate signal (see FIG. 6) and identify coupling between kinematics and flow (see FIG. 7).

In some instances, the fidelity of accelerometer derived breath intervals as assessed by the ARK system can be evaluated in a test setting. For example, the ARK engine can be configured to derive respiratory rate signals from accelerometer signals and true respiratory rate signals can be derived from synchronously collected volumetric flow signals from appropriate laboratory equipment. The ARK engine may use intervals between instantaneous phase landmarks on the accelerometer and flow signals in place of R-R intervals on electrocardiograms (as shown in FIG. 6). As described herein, a phase angle of 0 radians can be selected as a landmark for both flow and accelerometer signals, each of which can be resampled the respiratory rate at 1 Hz. To evaluate the fidelity of accelerometer-derived breath intervals, the cross-correlation between accelerometer-derived and flow-derived respiratory rate time series can be calculated.

In particular, FIG. 6 illustrates an exemplary respiratory rate sampling that can be performed by the ARK engine. For example, the top two curves (e.g., graphs 602 and 604) show a segment of the acceleration signal and its instantaneous phase. The respiratory rate samples derived from these intervals are shown at the bottom of FIG. 6 in plot 606. First, breath intervals (e.g., labeled as I₁ to I₄ in graph 604) are determined using consecutive occurrences of a phase angle (e.g., π radians, in this example). Next, a sampling rate for the respiratory rate (fᵣ) signal is chosen by the ARK engine as configured (e.g., 1 Hz in this example), without regard to mean respiratory rate or sampling frequency of the acceleration signal. For each sampling point, the ARK engine counts the number of breath intervals (nᵢ), including fractions, that occur within the time window extending from the previous sample to the next. For example, at time t₁,

$${n}_{{t}_{1}} = \left[\frac{a}{{I}_{2}}\right]$$

and at time t₂,

$${n}_{{t}_{2}} = \left[\frac{b}{{I}_{3}} + \frac{c}{{I}_{4}}\right] .$$

The respiratory rate (rᵢ) at each sampling point is calculated as

$${r}_{i} = \frac{{f}_{r} \times {n}_{i}}{2} .$$

In some embodiments, the ARK engine is configured to relate the phase of the flow signals and accelerometer signals. For example, the ARK engine is configured to calculate the relative frequency with which kinematic phase landmarks were distributed over the phase of the air flow cycle. In some embodiments, the ARK engine is configured to split the air flow cycle into 10 bins (e.g., bin width of

$$\frac{π}{5}$$

radians). In order to quantify synchronization, the ARK engine is configured to calculate the Shannon Entropy of the histograms:

$$S \left(x\right) = {\sum }_{j = 1}^{10} P \left({x}_{j}\right) ⋅ {log}_{2} P \left({x}_{j}\right)$$

Here, P(xⱼ) represents the probability (e.g., relative frequency) of a kinematic phase landmark occurring in the j^{th} bin of air flow phase. In some embodiments, this measurement may quantify instances of cardiopulmonary coupling. For instance, the Shannon entropy can range between a maximum of 3.32 bits (i.e., log210) signifying uniform distribution across 10 bins, and a minimum of 0 bits signifying localization to a single bin as shown in FIG. 7.

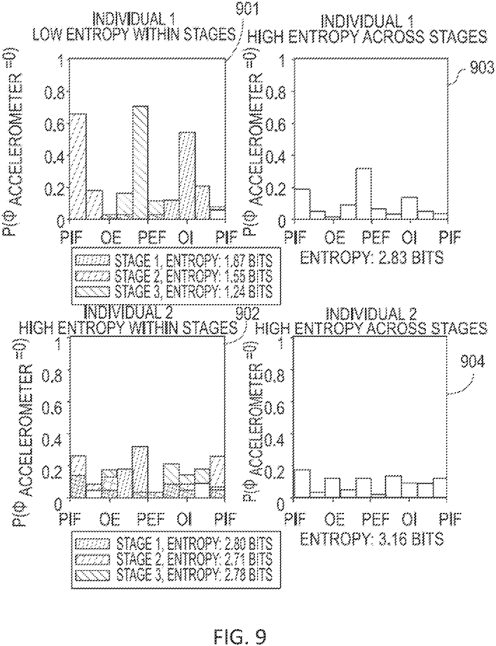

In FIG. 7, a graph depicted a method to measure kinematics flow coupling between signals using one example each of strong (top) and weak (bottom) coupling is presented. For example, when coupling is strong, landmarks from the phase of the acceleration signals (e.g., phase of 0 radians, in this scenario) are strongly localized to a particular portion of the phase of the flow cycle (e.g., mid-exhalation), resulting in low entropy (e.g., 0.35 bits in this example). When coupling is weak, landmarks are uniformly distributed over the phase of the flow phase, resulting in high entropy (e.g., 2.86 bits). In FIG. 7 (as well as FIG. 9 below), the following abbreviations and/or representations are used: PIF: Peak Inspiratory Flow; OE: Onset of Exhalation; PEF: Peak Expiratory Flow; OI: Onset of Inhalation; φ = Phase angle.

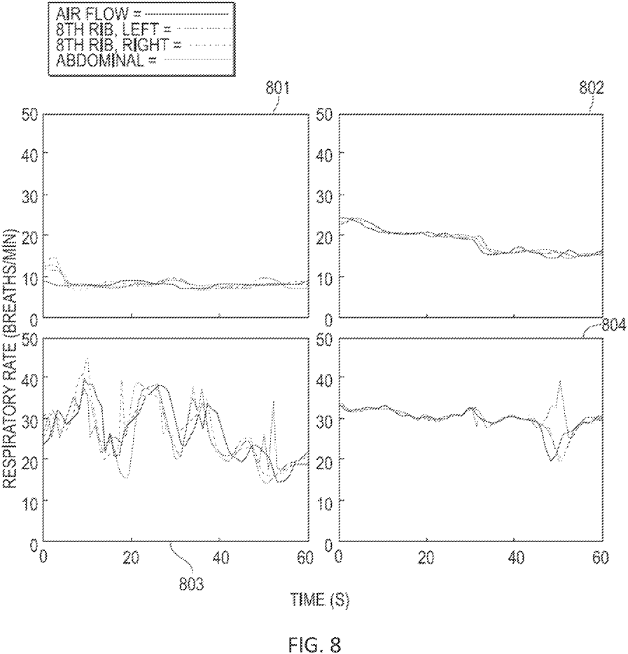

In conclusion, a strong relationship is found between respiratory rate time series derived from accelerometer signals and flow signals. In light of the variability that is observed in the kinematic-flow cycle synchronicity, the ARK engine can be configured to quantify the degree of agreement using the maximal cross-correlation value without regard for lag. Notably, cross-correlation coefficients as high as 0.94 were observed. Of note, the strength of this relationship was preserved despite trends, cyclical fluctuations or transient disturbances in true respiratory rate (e.g., see FIG. 8). In particular, FIG. 8 highlights the diversity of respiratory rate signals that are observed in a small sample. The rate signals plotted in the solid black line were derived by the ARK engine from air flow signals whereas dotted lines represent rate signals sampled from 8^{th} rib (left and right) and abdominal acceleration signals. Graph 801 represents a resting series with a very stable respiratory rate. Graphs 802-804 are all series obtained at exhaustion and show a variable recovery pattern. In graph 802, there is a smooth downward trend recovery in respiratory rate. Graph 803 shows a cyclical fluctuation of respiratory rate superimposed on a downward trend. In graph 804, very gradual recovery that is interrupted with a transient slow-down (e.g., sharp transient deceleration) is shown. In each of these instances, respiratory rate signals were reproduced with high fidelity in the accelerometer-derived respiratory rate signal using the ARK engine and/or method.

When analyzed across individuals and exercise states, landmarks from the phase of kinematic signals were uniformly distributed across the phase of the air flow cycle (see FIG. 9). The average Shannon entropy across the lower rib and abdominal sensors was 3.30, which is comparable to the entropy limit of 3.32 for a completely uniform distribution. Specifically, FIG. 9 illustrates the observation that kinematics and flow have a complex and diverse relationship which varies between and within individuals. Graphs 901 shows findings from an individual whose kinematics were strongly coupled with flow within a stage. In each stage, however, the kinematic phase landmarks localized to different areas of the air flow phase. As a result, the overall Shannon entropy (see graph 903) for that individual was higher (2.83 bits) than their stage-specific Shannon Entropy (1.87, 1.55, 1.24). Notably, the point of localization varied by exercise stage - occurring in mid exhalation, at peak inspiratory flow, and at onset of inhalation during rest, lactate threshold and maximal exercise respectively. The overall localization was poor (Shannon entropy of 2.83) when all breaths were considered without regard to exercise state. In some other individuals, no localization even within a stage was found.

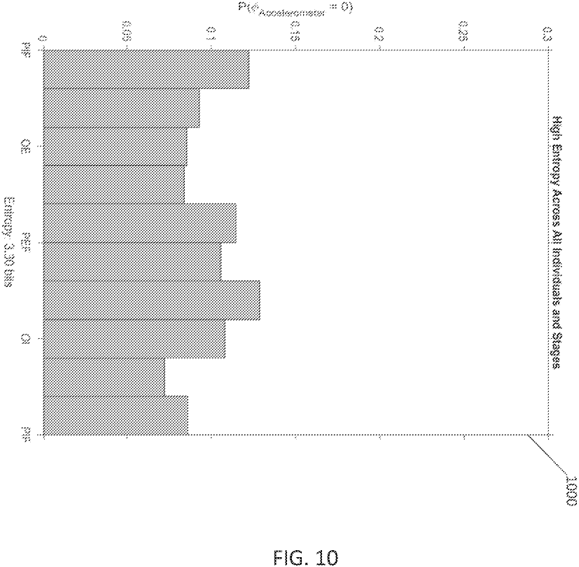

Likewise, graph 902 shows findings from an individual whose kinematics weakly coupled with flow even within a stage. Across individuals and exercises stages, therefore, the overall Shannon entropy (3.30 bits) was very close to the maximal possible value of 3.32 (see FIG. 10). Notably, graph 1000 in FIG. 10 displays results obtained from a lower rib sensor. Similar results were noted at all sensor locations.

Although the above describes an exemplary method for determining the degree of kinematic coupling, it is appreciated that other similar methods can be used to detect the degree of coupling between ARK sensor locations (e.g., thoraco-abdominal signal coupling) without departing from the scope of the disclosed subject matter.

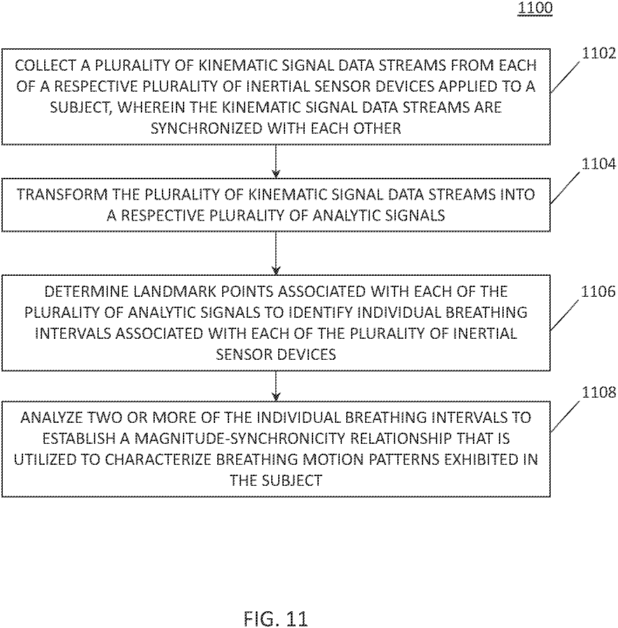

FIG. 11 is a flow chart illustrating an exemplary process or method 1100 for providing updated network slice information to a NSSF according to an embodiment of the subject matter described herein. In some embodiments, method 1100 depicted in FIG. 11 is an algorithm, program, or script (e.g., ARK engine 108 as shown in FIG. 1) stored in memory that when executed by a processor performs the steps recited in blocks 1102-1108. In some embodiments, the ARK engine may represent a list of steps (or changes in steps) embodied in a state machine (e.g., either via software code programming or via a set of rules).

In block 1102, method 1100 includes collecting a plurality of kinematic signal data streams from each of a respective plurality of inertial sensor devices applied to a subject. In some embodiments, a number of sensor devices are placed on a patient subject and subsequently synchronized with each other (i.e., such that the kinematic signal data streams are all synchronized). Notably, the plurality of sensor devices are communicatively coupled to a monitoring platform device via a wireless or wired connection. In some embodiments, the monitoring platform device is configured to execute an ARK engine that is responsible for managing the collection the kinematic signal the streams from the sensor devices over the connection.

In block 1104, method 1100 includes transforming the plurality of kinematic signal data streams into a respective plurality of analytic signals. Once the kinematic signal data streams are received by and stored on the monitoring platform device, the ARK engine is configured to apply a mathematical transform in order to find an analytic signal for each of the kinematic signal data streams. In some embodiments, the mathematical transform utilized by the ARK engine is a Hilbert transform. In addition, the ARK engine may also be configured to conduct a filtering process prior to the aforementioned transformation process. For example, the ARK engine may be configured to filter the kinematic signal data streams with digital signal processing in order to remove unwanted noise. In some embodiments, the filtered data can be further re-filtered for self-similarity and markers of clean data (e.g., thresholds of amplitude, variance, sample entropy, autocorrelations, and cross correlations).

In block 1106, method 1100 includes determining landmark points associated with each of the plurality of analytic signals to identify individual breathing intervals associated with each of the plurality of inertial sensor devices. In some embodiments, the ARK engine is configured to identify landmark points on the analytic signals. For example, the ARK engine may determine Hilbert cardinal points (HCPs) on analytic phase functions or phase angles. Using this information, the ARK engine is capable of extracting clean individual breathing intervals for further processing.

In block 1108, method 1100 includes analyzing two or more of the individual breathing intervals to establish a magnitude-synchronicity relationship that is utilized to determine a probability of a presence of a respiratory condition existing in the subject. In particular, the ARK engine is configured to conduct a post-processing pipeline on the individual breathing intervals obtained in block 1106. For example, the ARK engine can align a plurality of the individual breath intervals to assess the magnitude of motion of various signal data captured by two or more sensor devices (which are respectively located in different positions). More specifically, ARK engine capable of simultaneously comparing the magnitude of the synchronized signals captured by two or more sensor devices that are located in different positions on a patient subject’s chest and abdomen area. Based on this exhibited magnitude-synchronicity relationship, the ARK engine can utilize this data as input for a statistical model (e.g., such as a decision tree) that is capable of predicting the probability of the patient subject having an outcome of interest, such as a respiratory condition (e.g., labored breathing condition). For example, the statistical model can generate a numeric likelihood or probability ranging from 0% to 100% (i.e., 0 to 1.0). In some embodiments, the engine can be configured to issue an alert to a patient user, physician, and/or caretaker if the probability or the change in probability exceeds a predefined threshold. Alternatively, the ARK engine can be configured to instead produce a continuous display of signal data and/or probability data for interpretation by a physician as opposed to generating threshold driven alert.

Notably, after the landmarks are identified (e.g., see block 1106) using analytical signals or otherwise, the ARK engine can be adapted to utilize the identified landmarks in various ways. For example, the ARK engine can be configured to obtain a respiratory rate time series and subsequently characterize the RR time series using any of the number of established time series analysis methods. The ARK engine may also be configured to measure magnitudes of acceleration, both on a breath by breath basis and for a continuous signal strip as a whole. In particular, the ARK engine can be configured to quantify relationships between measured magnitudes between sensor device locations, both on a breath by breath basis and for a continuous signal strip as a whole. Further, the ARK engine can be configured to quantify the degree of synchronicity and phase relationships between the sensor device locations.

In other embodiments, the ARK engine can be configured to utilize the synchronized individual breathing intervals to objectively produce other information that is of interest to physicians and caretakers. For example, the ARK engine can produce regularly sampled respiratory rate time series data and subsequently conduct a time series analysis on the respiratory rate (e.g., mean, variance, entropy, etc.). As such, the average respiratory rate, metrics on stability and consistency of the respiratory rate, and feature detection for regular breathing patterns can also be assessed by the ARK engine. In addition, the acceleration amplitudes of interest, the acceleration ratios of interest, the classification of breath intervals as abdominal, thoracic, or mixed thoraco-abdominal can be achieved using the ARK engine processing.

In some embodiments, the ARK engine can also be used to calculate ratios of signal amplitudes in order to determine the functional difference between each sensor device’s analytic phase function (APF) and dominant APF on a per breath basis. Such data can also be used to average each functional difference to find a per-breath, per-sensor lag, that may be used for compartment synchronization.

The relationship between kinematic and air flow cycles has been found to be rich with complexity - varying between and within individuals. For practical breath-detection applications, the disclosed subject matter has an important implication - no landmark point is superior to any other in terms of optimizing alignment between the kinematic and air flow cycles. The phase angle of π radians is selected to identify each nadir in the accelerometer signals. Any other landmark (e.g., like the phase angle of 0 radians to identify each peak of the accelerometer signal) might be selected.

It has been demonstrated that the ARK signals can yield highly resolved respiratory rate time series from kinematic signals. This finding paves way for two types of analyses in the future. First, a large number of well-established mathematical operations can be used to characterize respiratory rate time series in any clinical setting. The regular sampling on a real-time axis also allows for meaningful analysis in the frequency domain. Second, well-defined breath intervals may allow breath-by-breath characterization of signal features, i.e., comparing magnitude-synchrony relationships between different sensor device locations within a single kinematic cycle. This is important to quantify patterns like respiratory alternans or opiate-induced ataxic breathing, where the breathing pattern varies from one breath to the next. Future advancements can build on this breath detection method to describe the clinical significance of a large and diverse set of metrics. The overarching vision is to create a set of novel respiratory vital signs that may be conveniently measured in any setting and improve medical decision making in common clinical scenarios.

One advantage afforded by the disclosed subject matter is that one or more embodiments are grounded in fundamentally sound analytic methods like the Hilbert transform, the analytic representation of a signal, Shannon entropy, and Berger’s interpolation algorithm. Notably, the disclosed subject matter does not rely on any arbitrary assumptions, decisions, thresholds or models. This enhances the reliability, reproducibility of the disclosed ARK system and method and its generalizability to other signals that record respiratory motion (e.g., impedance, inductance plethysmography etc.).

Accordingly, the disclosed subject matter describes a reliable and reproducible method and system capable of detecting individual breath cycles from respiratory kinematic signals. Despite a complex relationship between respiratory kinematics and air flow, the ARK method and system resulted in highly resolved respiratory rate time series. Notably, this will facilitate quantitative characterization of clinically significant breathing motion patterns in any care setting.

It will be understood that various details of the presently disclosed subject matter can be changed without departing from the scope of the presently disclosed subject matter. Furthermore, the foregoing description is for the purpose of illustration only, and not for the purpose of limitation.

### REFERENCES

All references listed in the instant disclosure, including but not limited to all patents, patent applications and publications thereof, scientific journal articles, and database entries are incorporated herein by reference in their entireties to the extent that they supplement, explain, provide a background for, and/or teach methodology, techniques, and/or compositions employed herein. The discussion of the references is intended merely to summarize the assertions made by their authors. No admission is made that any reference (or a portion of any reference) is relevant prior art. Applicant reserves the right to challenge the accuracy and pertinence of any cited reference.

1. Tobin MJ, Chadha TS, Jenouri G, Birch SJ, Gazeroglu HB, Sackner MA. Breathing patterns. 1. Normal subjects. Chest 1983;84:202-205.

2. Tobin MJ, Chadha TS, Jenouri G, Birch SJ, Gazeroglu HB, Sackner MA. Breathing patterns. 2. Diseased subjects. Chest 1983;84:286-294.

3. Braun SR. Respiratory Rate and Pattern. In: Walker HK, Hall WD, Hurst JW, editors. Clinical Methods: The History, Physical, and Laboratory Examinations, 3rd ed. Boston: Butterworths; 1990. at <http://www.ncbi.nlm.nih.gov/books/NBK365/>.

4. American Thoracic Society/European Respiratory Society. ATS/ERS Statement on respiratory muscle testing. Am J Respir Crit Care Med 2002;166:518-624.

5. Magder S. Bench-to-bedside review: ventilatory abnormalities in sepsis. Crit Care 2009;13:202.

6. Cohen CA, Zagelbaum G, Gross D, Roussos C, Macklem PT. Clinical manifestations of inspiratory muscle fatigue. Am J Med 1982;73:308-316.

7. Bouillon T, Bruhn J, Roepcke H, Hoeft A. Opioid-induced respiratory depression is associated with increased tidal volume variability. Eur J Anaesthesiol 2003;20:127-133.

8. Walker JM, Farney RJ, Rhondeau SM, Boyle KM, Valentine K, Cloward TV, Shilling KC. Chronic Opioid Use is a Risk Factor for the Development of Central Sleep Apnea and Ataxic Breathing. J Clin Sleep Med 2007;3:455-461.

9. Ermer SC, Farney RJ, Johnson KB, Orr JA, Egan TD, Brewer LM. An Automated Algorithm Incorporating Poincaré Analysis Can Quantify the Severity of Opioid-Induced Ataxic Breathing. Anesthesia & Analgesia 2020;130:1147-1156.

10. Liu H, Allen J, Zheng D, Chen F. Recent development of respiratory rate measurement technologies. Physiol Meas 2019;40:07TR01.

11. Mohr MA, Fairchild KD, Patel M, Sinkin RA, Clark MT, Moorman JR, Lake DE, Kattwinkel J, Delos JB. Quantification of periodic breathing in premature infants. Physiol Meas 2015;36:1415-1427.

12. Tinoco A, Mortara DW, Hu X, Sandoval CP, Pelter MM. ECG Derived Cheyne-Stokes Respiration and Periodic Breathing are Associated with Cardiorespiratory Arrest in Intensive Care Unit Patients. Heart Lung 2019;48:114-120.

13. Laveneziana P, Albuquerque A, Aliverti A, Babb T, Barreiro E, Dres M, Dubé B-P, Fauroux B, Gea J, Guenette JA, Hudson AL, Kabitz H-J, Laghi F, Langer D, Luo Y-M, Neder JA, O′Donnell D, Polkey MI, Rabinovich RA, Rossi A, Series F, Similowski T, Spengler CM, Vogiatzis I, Verges S. ERS statement on respiratory muscle testing at rest and during exercise. Eur Respir J 2019;53:1801214.

14. Borg G. Perceived exertion as an indicator of somatic stress. Scand J Rehabil Med 1970;2:92-98.

15. Seip RL, Snead D, Pierce EF, Stein P, Weltman A. Perceptual responses and blood lactate concentration: effect of training state. Med Sci Sports Exerc 1991;23:80-87.

16. Rosenblum null, Pikovsky null, Kurths null. Phase synchronization of chaotic oscillators. Phys Rev Lett 1996;76:1804-1807.

17. Berger RD, Akselrod S, Gordon D, Cohen RJ. An efficient algorithm for spectral analysis of heart rate variability. IEEE Trans Biomed Eng 1986;33:900-904.

18. Schäfer C, Rosenblum MG, Kurths J, Abel HH. Heartbeat synchronized with ventilation. Nature 1998;392:239-240.

19. Clark MT, Rusin CG, Hudson JL, Lee H, Delos JB, Guin LE, Vergales BD, Paget-Brown A, Kattwinkel J, Lake DE, Moorman JR. Breath-by-breath analysis of cardiorespiratory interaction for quantifying developmental maturity in premature infants. J Appl Physiol (1985) 2012;112:859-867.

20. MATLAB R2020b. Natick, Massachusetts: The MathWorks Inc.; 2020.

21. Harris PA, Taylor R, Thielke R, Payne J, Gonzalez N, Conde JG. Research electronic data capture (REDCap)--a metadata-driven methodology and workflow process for providing translational research informatics support. J Biomed Inform 2009;42:377-381.

22. Harris PA, Taylor R, Minor BL, Elliott V, Fernandez M, O′Neal L, McLeod L, Delacqua G, Delacqua F, Kirby J, Duda SN, REDCap Consortium. The REDCap consortium: Building an international community of software platform partners. J Biomed Inform 2019;95:103208.

23. Philip KEJ, Pack E, Cambiano V, Rollmann H, Weil S, O′Beirne J. The accuracy of respiratory rate assessment by doctors in a London teaching hospital: a cross-sectional study. J Clin Monit Comput 2015;29:455-460.

24. Latten GHP, Spek M, Muris JWM, Cals JWL, Stassen PM. Accuracy and interobserver-agreement of respiratory rate measurements by healthcare professionals, and its effect on the outcomes of clinical prediction/diagnostic rules. PLoS One 2019;14:e0223155.

25. Tulaimat A, Patel A, Wisniewski M, Gueret R. The validity and reliability of the clinical assessment of increased work of breathing in acutely ill patients. Journal of Critical Care 2016;34:111-115.

26. Verghese A, Charlton B, Kassirer JP, Ramsey M, Ioannidis JPA. Inadequacies of Physical Examination as a Cause of Medical Errors and Adverse Events: A Collection of Vignettes. The American Journal of Medicine 2015;128:1322-1324.e3.

27. Tulaimat A, Trick WE. DiapHRaGM: A mnemonic to describe the work of breathing in patients with respiratory failure. PLoS One 2017;12:.

28. Tulaimat A, Gueret RM, Wisniewski MF, Samuel J. Association Between Rating of Respiratory Distress and Vital Signs, Severity of Illness, Intubation, and Mortality in Acutely III Subjects. Respiratory Care 2014;59:1338-1344.

29. Reeves JJ, Ayers JW, Longhurst CA. Telehealth in the COVID-19 Era: A Balancing Act to Avoid Harm. J Med Internet Res 2021;23:e24785.

30. Gelfman DM. Will the Traditional Physical Examination Be Another Casualty of COVID-19? Am J Med 2021;134:299-300.

31. Hyman P. The Disappearance of the Primary Care Physical Examination-Losing Touch. JAMA Intern Med 2020;180:1417.

32. Fulcher BD, Little MA, Jones NS. Highly comparative time-series analysis: the empirical structure of time series and their methods. Journal of The Royal Society Interface 2013;10:20130048.

What is claimed is:

1. A method for analyzing respiratory kinematics, the method comprising:
collecting a plurality of kinematic signal data streams from each of a respective plurality of inertial sensor devices applied to a subject, wherein the kinematic signal data streams are synchronized with each other;
transforming the plurality of kinematic signal data streams into a respective plurality of analytic signals;
determining landmark points associated with each of the plurality of analytic signals to identify individual breathing intervals associated with each of the plurality of inertial sensor devices; and
analyzing two or more of the individual breathing intervals associated with at least two of the inertial sensor devices to establish a magnitude-synchronicity relationship that is utilized to characterize breathing motion patterns exhibited in the subject.

2. The method of claim 1, wherein signal noise is removed from the kinematic signal data streams via one or more filters prior to transforming into the analytic signals.

3. The method of claim 1, wherein each of the plurality of inertial sensor devices is positioned in a unique and separate location on the torso of the subject.

4. The method of claim 1, wherein an instantaneous phase angle from each of the analytic signals is used to determine the landmark points.

5. The method of claim 1, wherein the magnitude-synchronicity relationship is provided as input to a statistical model that is configured to generate the probability.

6. The method of claim 1, wherein each of the plurality of kinematic signal data streams is collected via wireless communications.

7. The method of claim 1, wherein the magnitude-synchronicity relationship is defined by at least a comparison of magnitudes of motion exhibited by the analytic signals associated with two or more inertial sensor devices.

8. The method of claim 1, wherein the breathing motion patterns are utilized to determine a probability of a presence of a respiratory condition existing in the subject.

9. The method of claim 1, wherein the landmark points are utilized to derive a respiratory rate time series.

10. The method of claim 1, wherein identifying individual breathing intervals includes identifying breathing intervals on a breath by breath basis or on a continuous signal strip basis.

11. The method of claim 1, wherein the magnitude-synchronicity relationship is defined by a quantification of the degree of synchronicity and phase relationships exhibited by the analytic signals associated with two or more inertial sensor devices.

12. A system for analyzing respiratory kinematics, the system comprising:
a plurality of inertial sensor devices applied to a subject;
a monitoring platform device including at least one processor and a memory; and
an analysis of respiratory kinematics (ARK) engine stored in the memory and implemented by the at least one processor that is configured for collecting a plurality of kinematic signal data streams from each of the plurality of inertial sensor devices, wherein the kinematic signal data streams are synchronized with each other, transforming the plurality of kinematic signal data streams into a respective plurality of analytic signals, determining landmark points associated with each of the plurality of analytic signals to identify individual breathing intervals associated with each of the plurality of inertial sensor devices, and analyzing two or more of the individual breathing intervals associated with at least two of the inertial sensor devices to establish a magnitude-synchronicity relationship that is utilized to characterize breathing motion patterns exhibited in the subject.

13. The system of claim 12, wherein signal noise is removed from the kinematic signal data streams via one or more filters prior to transforming into the analytic signals.

14. The system of claim 12, wherein each of the plurality of inertial sensor devices is positioned in a unique and separate location on the torso of the subject.

15. The system of claim 12, wherein an instantaneous phase angle from each of the analytic signals is used to determine the landmark points.

16. The system of claim 12, wherein the magnitude-synchronicity relationship is provided as input to a statistical model that is configured to generate the probability.

17. The system of claim 12, wherein each of the plurality of kinematic signal data streams is collected via wireless communications.

18. The system of claim 12, wherein the magnitude-synchronicity relationship is defined by at least a comparison of magnitudes of motion exhibited by the analytic signals associated with two or more inertial sensor devices.

19. The system of claim 12, wherein the breathing motion patterns are utilized to determine a probability of a presence of a respiratory condition existing in the subject.

20-22. (canceled)

23. A non-transitory computer readable medium having stored thereon executable instructions that when executed by a processor of a computer control the computer to perform steps comprising:
collecting a plurality of kinematic signal data streams from each of a respective plurality of inertial sensor devices applied to a subject, wherein the kinematic signal data streams are synchronized with each other;
transforming the plurality of kinematic signal data streams into a respective plurality of analytic signals;
determining landmark points associated with each of the plurality of analytic signals to identify individual breathing intervals associated with each of the plurality of inertial sensor devices; and
analyzing two or more of the individual breathing intervals associated with at least two of the inertial sensor devices to establish a magnitude-synchronicity relationship that is utilized to characterize breathing motion patterns exhibited in the subject.

24-33. (canceled)
